(19) United States
(12) Patent Application Publication
(10) Pub. No.: US 2002/0077323 A1
(43) Pub. Date: Jun. 20, 2002

(54) Use of delta opioid receptor ligands and serotonin reuptake inhibitors in the treatment of chemical dependencies

(76) Inventors: Stafford McLean, Stonington, CT (US); Spiros Liras, Stonington, CT (US); Stanton F. Mchardy, Coventry, RI (US)

Correspondence Address: Paul H. Ginsburg, Pfizer Inc, 20th Floor, 235 East 42nd Street, New York, NY, 10017-5755, US

(21) Appl. No.: 09/901,362

(22) Filed: Jul. 9, 2001

Related U.S. Application Data
(60) Provisional application No. 60/217,548, filed on Jul. 12, 2000.

(51) Int. Cl. A61K 31/5377; A61K 31/53; A61K 31/522; A61K 31/498; A61K 31/517; A61K 31/454; A61K 31/4709

(52) U.S. Cl. 514/235.5; 514/234.5; 514/242; 514/248; 514/249; 514/255.05; 514/263.22; 514/266.22; 514/314; 514/318; 514/326

(57) ABSTRACT

A method for treating a chemical dependency that involves administering an amount of a delta opioid receptor ligand and a serotonin reuptake inhibitor, the amounts being effective in the combination to treat the dependency, wherein the delta opioid receptor ligand is selected from the group consisting of:

a) a compound of the formula

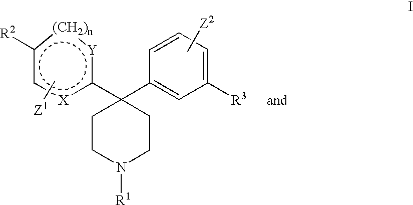

and

b) a compound of the formula

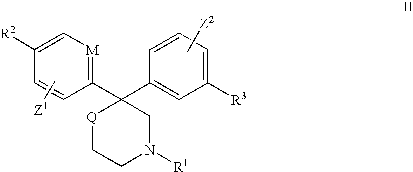

wherein X, Y, M, n, Z¹, Z², R¹, R², and R³ are defined as described herein.

### FIELD OF THE INVENTION

This invention relates to the treatment of chemical dependencies using combination therapy.

### BACKGROUND OF THE INVENTION

In the study of opioid biochemistry, a variety of endogenous opioid compounds and non-endogenous opioid compounds has been identified. In this effort, significant research has been focused on understanding the mechanism of opioid drug action, particularly as it relates to cellular and differentiated tissue opioid receptors.

Opioid drugs are typically classified by their binding selectivity in respect of the cellular and differentiated tissue receptors to which a specific drug species binds as a ligand. These receptors include mu (μ), delta (δ) and kappa (κ) receptors.

At least three subtypes of opioid receptors (mu, delta and kappa) are described and documented in the scientific literature. All three receptors are present in the central and peripheral nervous systems of many species including man. Activation of delta receptors produces antinociception in rodents and can induce analgesia in man, in addition to influencing motility of the gastrointestinal tract. (See Burks, T. F. (1995) in “The pharmacology of Opioid Peptides”, edited by Tseng, L. F., Harwood Academic Publishers).

The well known narcotic opiates such as morphine and its analogs are selective for the opioid mu receptor. Mu receptors mediate analgesia, respiratory depression, and inhibition of gastrointestinal transit. Kappa receptors mediate analgesia and sedation.

The existence of the opioid delta receptor is a relatively recent discovery which followed the isolation and characterization of endogenous enkephalin peptides, which are ligands for the delta receptor. Research in the past decade has produced significant information about the delta receptor, but a clear picture of its function has not yet emerged. Delta receptors mediate analgesia, but do not appear to inhibit intestinal transit in the manner characteristic of mu receptors.

U.S. Pat. No. 4,816,586, which issued on Mar. 28, 1989 to P. S. Portoghese, refers to various delta opioid receptor antagonists. These compounds are described as possessing a unique opioid receptor antagonist profile, and include compounds that are highly selective for the delta opioid receptor.

U.S. Pat. No. 4,518,711, which issued May 21, 1985 to V. J. Hruby et al., describes cyclic, conformationally constrained analogs of enkephalins. These compounds include both agonists and antagonists for the delta receptor, and are said to induce pharmacological and therapeutic effects, such as analgesia in the case of agonist species of such compounds. The antagonist species of the disclosed compounds are suggested to be useful in the treatment of schizophrenia, Alzheimer's disease, and respiratory and cardiovascular functions.

S. Goenechea, et al., in “Investigation of the Biotransformation of Meclozine in the Human Body,” J. Clin. Chem. Clin. Biochem., 1988, 26(2), 105-15, describe the oral administration of a polyaryl piperazine compound in a study of meclozine metabolization in human subjects.

In “Plasma Levels, Biotransformation and Excretion of Oxatomide in Rats, Dogs, and Man,” Xenobiotica, 1984, 15(6), 445-62, Meuldermans, W., et al. refer to a metabolic study of plasma levels, biotransformation, and excretion of oxatomide.

T. Iwamoto, et al, in “Effects of KB-2796, A New Calcium Antagonist, and Other Diphenylpiperazines on [³H]nitrendipine Binding”, Jpn. J. Pharmacol., 1988, 48(2), 241-7, describe the effect of a polyaryl piperazine as a calcium antagonist.

K. Natsuka, et al, in “Synthesis and Structure-Activity Relationships of 1-Substituted 4-(1,2-Diphenylethyl)piperazine Derivatives Having Narcotic Agonist and Antagonist Activity,” J. Med. Chem., 1987, 30 (10), 1779-1787, refer to racemates and enantiomers of 1-substituted 4-[2-(3-hydroxyphenyl)-1-phenylethyl]piperazine derivatives.

European Patent Application No. 458,160, published on Nov. 27, 1991, refers to certain substituted diphenylmethane derivatives as analgesic and antiinflammatory agents, including compounds wherein the methylene bridging group (linking the two phenyl moieties) is substituted on the methylene carbon with a piperidinyl or piperazinyl group.

South African Patent Application No. 8604522, which was published on Dec. 12, 1986, refers to certain N-substituted arylalkyl and aryl-alkylene substituted amino-heterocyclic compounds, including piperidine derivatives, as cardiovascular, antihistamine, and anti-secretory agents.

European Patent Application No. 133,323, published on Feb. 20, 1985, refers to certain diphenylmethyl piperazine compounds as non-sedative antihistamines.

PCT/IB99/01914, filed Dec. 1, 1999, refers to 3,3-biarylpiperidine and 2,2-biarylmorpholine derivatives having the ability to selectively bind to delta opioid receptors. WO 00/14066, published Mar. 16, 2000, refers to 4,4-biarylpiperidine derivatives having the ability to selectively bind to delta opioid receptors. U.S. patent application Ser. No. 09/503,679, filed Feb. 14, 2000, refers to 4-phenyl-4-heteroarylpiperidine derivatives having the ability to selectively bind to delta opioid receptors. The compounds described in these applications are referred to as useful in the treatment of chemical dependencies and addictions.

Serotonin reuptake inhibitors have also been referred to as useful in the treatment of chemical dependencies and addictions. U.S. Pat. No. 5,130,338, issued Jul. 14, 1992, refers to the use of sertraline in treating chemical dependencies and addictions. International Application Publication No. WO96109047, published Mar. 28, 1996, refers to the use of citalopram, zimelidine, fluoxetine, and fluvoxamine in reducing alcohol consumption in mammals, and describes a combination of an opioid antagonist and a serotonin reuptake inhibitor for treating alcoholism and alcohol dependence. U.S. Pat. No. 6,001,848, issued Dec. 14, 1999 refers to administration of a dopaminergic or opioidergic compound to treat alcoholism; the patent states that a serotonin reuptake inhibitor may also be administered.

### SUMMARY OF THE INVENTION

The invention relates to a method for treating a chemical dependency comprising administering an amount of a delta opioid receptor ligand and a serotonin reuptake inhibitor, said amounts being effective in said combination to treat said dependency, wherein said delta opioid receptor ligand is selected from the group consisting of:

a) a compound of the formula

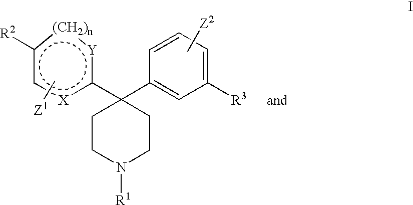

and

b) a compound of the formula

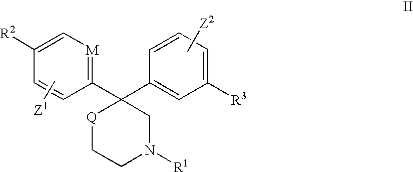

wherein

X and Y are selected, independently, from oxygen, nitrogen, sulfur and CH, with the proviso that the ring in compound I containing X and Y must be aromatic and with the proviso that X and Y in compound I cannot both be either oxygen or sulfur;

Q is oxygen or CH₂;

M is CH or N;

n is zero or one;

R¹ is hydrogen, (C₀-C₈)alkoxy-(C₀-C₈)alkyl-, wherein the total number of carbon atoms is eight or less, aryl, aryl-(C₁-C₈)alkyl-, heteroaryl, heteroaryl-(C₁-C₈)alkyl-, heterocyclic, heterocyclic-(C₁-C₈)alkyl, (C₃-C₇)cycloalkyl-, or (C₃-C₇)cycloalkyl-(C₁-C₈)alkyl, wherein said aryl and the aryl moiety of said aryl-(C₁-C₈)alkyl- are selected, independently, from phenyl and naphthyl, and wherein said heteroaryl and the heteroaryl moiety of said heteroaryl-(C₁-C₈)alkyl- are selected, independently, from pyrazinyl, benzofuranyl, quinolyi, isoquinolyi, benzothienyl, isobenzofuryl, pyrazolyl, indolyl, isoindolyl, benzimidazolyl, purinyl, carbazolyl, 1,2,5-thiadiazolyl, quinazolinyl, pyridazinyl, pyrazinyl, cinnolinyl, phthalazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidinyl, pyrazolopyrimidinyl, oxazolyl, oxadiazolyl, isoxazolyl, thiazolyl, isothiazolyl, furanyl, pyrazolyl, pyrrolyi, tetrazolyi, triazolyl, thienyl, imidazolyl, pyridinyl, and pyrimidinyl; and wherein said heterocyclic and the heterocyclic moiety of said heterocyclic-(C₁-C₈)alkyl- are selected from saturated or unsaturated nonaromatic monocyclic or bicyclic ring systems, wherein said monocyclic ring systems contain from four to seven ring carbon atoms, from one to three of which may optionally be replaced with 0, N or S, and wherein said bicyclic ring systems contain from seven to twelve ring carbon atoms, from one to four of which may optionally be replaced with 0, N or S; and wherein any of the aryl, heteroaryl or heterocyclic moieties of R¹ may optionally be substituted with from one to three substituents, preferably with one or two substituents, independently selected from halo, (C₁-C₆)alkyl optionally substituted with from one to seven (preferably with from zero to four) fluorine atoms, phenyl, benzyl, hydroxy, acetyl, amino, cyano, nitro, (C₁-C₆)alkoxy, (C₁-C₆)alkylamino and [(C₁-C₆)alkyl]₂amino, and wherein any of alkyl moieties in R¹ (, the alkyl moieties of alkyl, alkoxy or alkylamino groups) may optionally be substituted with from one to seven (preferably with from zero to four) fluorine atoms;

R² is hydrogen, aryl, halo, heteroaryl, heterocyclic, SO₂R⁴, COR⁴, CONR⁵R⁶, COOR⁴, or C(OH)R⁵R⁶ wherein each of R4, R⁵ and R⁶ is defined, independently, as R¹ is defined above, or R⁵ and R6, together with the carbon or nitrogen to which they are both attached, form a three to seven membered saturated ring containing from zero to three heterocarbons selected, independently, from O, N and S, and wherein said aryl, heteroaryl, and heterocyclic are defined as such terms are defined above in the definition of R¹, and wherein any of the aryl, heteroaryl and heterocyclic moieties of R² may optionally be substituted with from one to three substituents, preferably with one or two substituents, independently selected from halo, (C₁-C₆)alkyl optionally substituted with from one to seven (preferably with from zero to four) fluorine atoms, phenyl, benzyl, hydroxy, acetyl, amino, cyano, nitro, (C₁-C₆)alkoxy optionally substituted with from one to seven (preferably with from zero to four) fluorine atoms, (C₁-C₆)alkylamino and [(C₁-C₆)alkyl]₂amino;

R³ is hydroxy, —(C₁-C₆)alkyl-OH, —OC(=O)R⁷, —(C₁-C₆)alkyl-(C₁-C₆)alkoxy, NHSO₂R⁷, C(OH)R⁷R⁸, halo, or heteroaryl as defined for R¹ above or CONHR⁷, wherein R⁷ and R⁸ are the same or different and are selected from hydrogen, (C₁-C₄)alkyl, (C₁-C₄)alkoxy and (C₁-C₄)alkoxy-(C₁-C₄)alkyl having a total of 4 or less carbon atoms, and wherein any of the alkyl moieties of R⁷ and R⁸ may optionally be substituted with from one to seven (preferably with from zero to four) fluorine atoms; and

Z¹ and Z² are independently hydrogen, halo or (C₁-C₅)alkyl;

with the proviso that there are no two adjacent ring oxygen atoms and no ring oxygen atom adjacent to either a ring nitrogen atom or a ring sulfur atom in any of the heterocyclic or heteroaryl moieties of formula 1, or 11;

and the pharmaceutically acceptable salts of such compounds.

In a preferred embodiment, the delta opioid receptor ligand is a receptor antagonist.

Preferred compounds of the formula I for use in the invention include those wherein n is zero or one; X and Y are both nitrogen or X is nitrogen and Y is CH or oxygen; R¹ is benzyl, cyclopropylmethyl, 2-pyridyl, 4-fluoro-2-pyridyl, pyrimidyl, 2-methylpentyl, 3-phenylpropyl, 2-ethoxyethyl or 3,5,5-trimethylhexyl; R² is CON(CH₂CH₃)₂, CON(CH₃)₂, CON(CH₂CH₃)CH₃, C(OH)(CH3)₂, C(OH)(CH₂CH₃)₂, 3,3-dimethyloxazoline, 3,3-diethyloxazoline, benzoxazole, tetrazole or 3,5-dimethylpyrazole; and R³ is OH, CONH₂, fluoro, bromo, chloro, iodo, or NHSO₂R⁷. In one embodiment of the preferred compounds, n is zero, Y is CH, and R³ is OH or CONH₂.

Other preferred compounds of the formula I for use in the invention include those wherein n is 1, X and Y are CH, R¹ is cyclopropylmethyl, 3-cyclohexylpropyl, 2-phenylethyl, 2-methylpentyl, p-methylbenzyl, 2,2,2-trifluoroethyl, or 1-methylpentyl.

Other examples of preferred compounds of the formula I for use in the invention include those wherein n is 1, X and Y are CH, R² is diethyl amide, methyl ethyl amide, a diethyl carbinol, tetrazole, or pyrazole.

Other examples of preferred compounds of the formula I for use in the invention include those wherein n is 1, X and Y are CH and R³ is hydroxy, fluoro, CONH₂, NHSO₂CH₃, or methoxy.

Preferred compounds of the formula II for use in the invention include those wherein Q is CH₂.

Other preferred compounds of the formula II for use in the invention are those wherein X is CH.

Other preferred compounds of the formula II for use in the invention are those wherein R³ is OH, CONH₂, or fluoro.

Other preferred compounds, particularly of the formula II for use in the invention, are those wherein R2 is selected from C(OH)(C₂H₆)₂, CON(C₂H₆)₂, CONCH₃(C₂H₆) and the following cyclic groups:

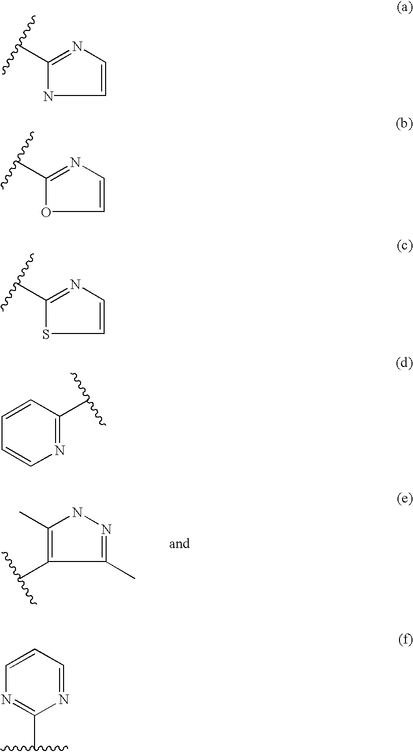

Other preferred compounds, particularly of the formula II, for use in the invention are those wherein Z¹ and Z² are selected, independently, from hydrogen and fluorine.

Other preferred compounds, particularly of the formula II, for use in the invention are those wherein R¹ is selected from allyl, cyclopropylmethyl, methyl, 2,2,2-trifluoroethyl, methallyl, isopropyl, 2-pyridinyl, 2-pyrimidinyl and

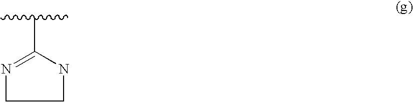

Examples of other embodiments of compounds used in the present invention are the following:

compounds of the formula II wherein Q is oxygen and M is CH;

compounds of the formula II wherein Q is oxygen and M is N;

compounds of the formula II wherein Q is oxygen, M is CH and R is OH, CONH₂, or fluoro;

compounds of the formula II wherein Q is CH₂, M is N, and R³ is OH, CONH₂, or fluoro;

compounds of the formula II wherein Q is CH₂, M is N, R ³ is OH, CONH₂, or fluoro, and R² is selected from C(OH)(C₂H₆)₂, CON(C₂H₆)₂ and one of cyclic groups (a)-(f) depicted above;

compounds of the formula II wherein Q is oxygen, M is N, R³ is OH, CONH₂, or fluoro, and R² is selected from C(OH)(C₂H₆)₂, CON(C₂H₆)₂ and one of cyclic groups (a)-(f) depicted above;

compounds of the formula II wherein Q is oxygen, M is CH, R³ is OH, CONH₂ or fluoro, Z¹ and Z² or selected, independently, from hydrogen and fluoro, and R¹ is selected from allyl, cyclopropylmethyl, methyl, methallyl, isopropyl, 2-pyridinyl, 2-pyrimidinyl and cyclic group (9) depicted above; and

compounds of the formula II wherein Q is oxygen, M is N, R³ is OH, CONH₂ or fluoro, Z¹ and Z² are selected, independently, from hydrogen and fluoro, and R¹ is selected from allyl, cyclopropylmethyl, methyl, methallyl, isopropyl, 2-pyridinyl, 2-pyrimidinyl and cyclic group (g) depicted above.

Any serotonin reuptake inhibitor can be used to practice the invention, including, without limitation, fluvoxamine, sertraline, citalopram, fluoxetine, paroxetine, imipramine, zimelidine, vanlafaxine, dapoxetine, and nefazodone.

In an alternative embodiment, the present invention relates to a pharmaceutical composition for the treatment of a chemical dependency wherein the composition comprises amounts of a delta opioid receptor ligand and a serotonin reuptake inhibitor, said amounts being effective in said combination to treat said dependency, wherein said delta opioid receptor ligand is selected from the group consisting of compounds I and II.

### DETAILED DESCRIPTION OF THE INVENTION

All patents, patent publications, and literature references cited herein are hereby incorporated by reference.

The term “chemical dependency,” as used herein, means any abnormal craving or desire for, addiction to, or abuse of, a substance. Such substances are generally administered to the dependent individual by oral, parenteral, nasal or by inhalation means. Examples of chemical dependencies treatable by the method of the present invention include dependencies on alcohol, nicotine, cocaine, heroin, phenobarbital, and benzodiazepines. Dependencies treatable by the present invention can be of either a physical and psychological nature.

The term “treatment”, as used herein, means reducing or alleviating the relevant dependency.

Unless otherwise indicated, the alkyl groups referred to herein, as well as the alkyl moieties of other groups referred to herein (e.g., alkoxy), may be linear or branched, and they may also be cyclic (e.g., cyclopropyl, cyclobutyl, cyclopentyl, or cyclohexyl) or be linear or branched and contain cyclic moieties.

The term “alkoxy”, as used herein, means “—O-alkyl”, wherein “alkyl” is defined as above.

The term “alkylene”, as used herein, means an alkyl group having two available binding sites (i.e., -alkyl-, wherein alkyl is defined as above).

Unless otherwise indicated, “halo” and “halogen”, as used herein, refer to fluorine, bromine, chlorine or iodine.

Compounds of the formula I or II may have chiral centers and therefore may exist in different enantiomeric and diastereomic forms. This invention relates to all optical isomers and all other stereoisomers of compounds of the formula I or II, and to all racemic and other mixtures thereof, and to all pharmaceutical compositions and methods of treatment defined above that contain or employ such isomers or mixtures.

Formulae I and II above include compounds identical to those depicted but for the fact that one or more hydrogen or carbon atoms are replaced by isotopes thereof.

Formulae I and II are intended to encompass any tautomers of the compounds.

Methods for making the delta opioid receptor ligands described above are disclosed in the above-listed patents and published patent applications, incorporated by reference herein. In particular, methods of making compounds of formula I are disclosed in US patent application Ser. No. 09/503,679, filed Feb. 14, 2000 and WO 00114066, published Mar. 16, 2000; and methods of making compounds of formula II are disclosed in PCT/IB99/01914, filed Dec. 1, 1999.

Methods for making the serotonin reuptake inhibitors are well-known in the art. Furthermore, serotonin reuptake inhibitors are commercially available. Fluoxetine, for example, is commercially available, and may be prepared according to methods described in U.S. Pat. No. 4,314,081. Sertraline is commercially available, and may be prepared as described in U.S. Pat. No. 4,536,518.

Combination treatments according to the invention can be administered as part of the same pharmaceutical composition, or the active agents can be administered separately as part of an appropriate dose regimen designed to obtain the benefits of the combination therapy.

Acid addition salts of the agents employed in the invention can be prepared in a conventional manner by treating a solution or suspension of the corresponding free base with one chemical equivalent of a pharmaceutically acceptable acid. Conventional concentration or crystallization techniques can be employed to isolate the salts. Illustrative of suitable acids are acetic, lactic, succinic, maleic, tartaric, citric, gluconic, ascorbic, benzoic, cinnamic, fumaric, sulfuric, phosphoric, hydrochloric, hydrobromic, hydroiodic, sulfamic, sulfonic acids such as methanesulfonic, benzene sulfonic, p-toluenesulfonic, and related acids.

The compounds and their pharmaceutically acceptable salts may be administered alone or in combination with pharmaceutically acceptable carriers, in either single or multiple doses. The active compounds may be formulated for oral, buccal, transdermal (e.g., patch), intranasal, parenteral (., intravenous, intramuscular or subcutaneous) or rectal administration or in a form suitable for administration by inhalation or insufflation. Suitable pharmaceutical carriers include inert solid diluents or fillers, sterile aqueous solutions, oils (e.g., peanut oil, sesame oil) and various organic solvents. The pharmaceutical compositions can be readily administered in a variety of dosage forms such as tablets, powders, lozenges, emulsions, oil soft gels, syrups, injectable solutions and the like. These pharmaceutical compositions can, if desired, contain additional ingredients such as flavorings, binders, excipients and the like. Thus, for purposes of oral administration, tablets containing various excipients such as sodium citrate, calcium carbonate and calcium phosphate may be employed along with various disintegrants such as starch, methylcellulose, alginic acid and certain complex silicates, together with binding agents such as polyvinylpyrrolidone, sucrose, gelatin and acacia. Additionally, lubricating agents such as magnesium stearate, sodium lauryl sulfate and talc are often useful for tabletting purposes. Solid compositions of a similar type may also be employed as fillers in soft and hard filled gelatin capsules. Preferred materials for this include lactose or milk sugar and high molecular weight polyethylene glycols. When aqueous suspensions or elixirs are desired for oral administration, the essential active ingredient therein may be combined with various sweetening or flavoring agents, coloring matter or dyes and, if desired, emulsifying or suspending agents, together with diluents such as water, ethanol, propylene glycol, glycerin and combinations thereof.

For parenteral administration, solutions containing the active compound in sesame or peanut oil, aqueous propylene glycol, or in sterile aqueous solution may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. The sterile aqueous media employed are all readily available by standard techniques known to those skilled in the art.

The effective dosages for the delta opioid receptor ligands and serotonin reuptake inhibitors employed in the method of this invention will depend on the intended route of administration and factors such as the age and weight of the patient, as generally known to a physician. The dosages will also depend on the particular condition to be treated and will generally range from about 0.001 to about 500 mg/kg body weight of the patient per day, with administration carried out in single or divided dosages. The delta opioid receptor ligand is preferably administered in a daily oral or intravenous dose of from 0.001 to 50 mg/kg body weight, preferably 0.1 to 20 mg/kg. The SSRI, e.g., sertraline, is preferably administered in a dosage of from about 12.5 mg/day to about 500 mg/day, more preferably from about 25 mg/day to about 200 mg/day.

The ability of the compounds of formula I or II to bind to the delta opioid receptor and the functional activity of the compounds at such receptor can be determined as described below. Binding to the delta opioid receptor can be determined using procedures well known in the art, such as those referred to by Lei Fang et al., J. Pharm. Exp. Ther., 268, 1994, 836-846 and Contreras et al., Brain Research, 604, 1993, 160-164.

In one embodiment, the delta opioid receptor ligand is administered in an amount effective to treat substance abuse, and the SSRI is administered in an amount effective to treat depression.

In the description of binding and functional assays that follows, the following abbreviations and terminology are used.

DAMGO is [D-Ala2,N-MePhe4,Gly5-ol]enkephalin).

U69593 is ((5a, 7a, 8b)-(+)-N-methyl-N-(7-[1-pyrrolidinyl]-1-oxasipro[4,5]dec-8-yl)-benzeneacetamide).

SNC-80 is (+)4-[(αR)-α((2S,5R)4-allyl-2,5dimethyl-1-piperazinyl)-3-methoxybenzyl]-N, N-diethylbenzamide.

nor BNI is nor-binaltorphimine.

CTOP is 1,2-Dithia-5,8,11,14,17-pentaazacycloeicosane, cyclic peptide derivative DPDPE is [D-en2,D-Pen5]enkephalin).

[3H]-SNC80 was prepared by Amersham International.

Delta receptor binding assays can be performed in a stable line of CHO cells expressing the human delta receptor. The binding assay can be carried out at 25° C. for 120 minutes in 50 mM Tris (pH 7.4) buffer. [C³H]-SNC-80 can be used to label delta receptor binding sites. The protein concentration can be approximately 12.5 μg/well. Non-specific binding can be defined with 10 μM naltrexone.

The binding reaction can be terminated by rapid filtration through glass fibre filters, and the samples can be washed with ice-cold 50 mM Tris buffer (pH 7.4).

Opioid activity is studied in two isolated tissues, the mouse deferens (MVD)(6) and the guinea-pig myentric plexus with attached longitudinal muscle (GPMP) (μ and k).

MVD (DC1 strain, Charles River, 25-35 g) are suspended in 15 ml organ baths containing Mg⁺⁺ free Krebs' buffer of the following composition (mM): NaCl, 119; KCI, 4.7; NaHCO₃, 25; KH₂PO₄, 1.2; CaCI₂, 2,5 and glucose, 11. The buffer is gassed with 95% 0₂ and 5% CO₂. The tissues are suspended between platinum electrodes, attached to an isometric transducer with 500 mg tension and stimulated with 0.03 Hz pulses of 1-msec pulse-width at supramaximal voltage. IC₅₀ values are determined by the regression analysis of concentration-response curves for inhibition of electrically-induced contractions in the presence of 300 nM of the mu-selective antagonist CTOP.

The following procedure can be used to determine the activity of the therapeutic agents employed in this invention as ligands of delta opioid receptors.

Cell Culture: Chinese hamster ovary cells expressing the human delta opioid receptor are passaged twice weekly in Hamis F-12 media with L-glutamine containing 10% fetal bovine serum and 450 μg/mL hygromycin. Cells are prepared for assays 3 days prior to the experiment. 15 mL of 0.05% trypsin/EDTA is added to a confluent triple flask, swirled and decanted to rinse. 15 mL of 0.05% trypsin/EDTA is again added, and the flask is placed into a 37C incubator for 2 minutes. Cells are removed from the flask by banking, and supernatant poured off into a 50 mL tube. 30 mL of media is then added to the flask to stop the action of the trypsin, and then decanted into the 50 mL tube. Tube is then centrifuged for 5 minutes at 1000 rpm, media decanted, and the pellet resuspended into 10 mL of media. Viability of the cells is assessed using trypan blue, the cells counted and plated out into 96 well poly-D-lysine coated plates at a density of 7,500 cells/well.

Antagonist Test Plate: Cells plated 3 days prior to assay are rinsed twice with PBS. The plates are placed into a 37C water bath. 50 μL of assay buffer (PBS, dextrose 1 mg/mL, 5 mM MgC12, 30 mM HEPES, 66.7 μg/mL of IBMX) is then added to designated wells. Fifty microliters of appropriate drug is then added to designated wells, and timed for 1 minute. Fifty microliters of 10 μM forskolin+0.4nM DPDPE (final assay concentration is 5 μM forskolin, 0.2nM DPDPE) is then added to appropriate wells, and timed for 15 minutes. The reaction is stopped by the addition of 10 μL of 6N perchloric acid to all wells. To neutralize, 13 μL of 5N KOH is added to all wells, and to stabilize 12 μL of 2M Tris, pH 7.4 is added to all wells. Mix by shaking on an orbital shaker for 10 minutes, and centrifuge at setting 7 for 10 minutes. Aliquot into a 3H plate.

Agonist Test Plate: Cells plated 3 days prior to assay are rinsed twice with PBS. The plates are placed into a 37° C. water bath. Fifty microliters of assay buffer (PBS, dextrose 1 mg/mL, 5 mM MgCl₂, 30 mM HEPES, 66.7 μg/mL of IBMX) is then added to designated wells. Fifty microliters of appropriate drug+10 μM forskolin (final assay concentration is 5 μM forskolin) is then added to all wells, and timed for 15 minutes. The reaction is then stopped by the addition of 10 μL of 6N perchloric acid to all wells. To neutralize, 13 μp of 5N KOH is added to all wells, and to stabilize 12 μL of 2M Tris, pH 7.4 is added to all wells. Mix by shaking on an orbital shaker for 10 minutes, and centrifuge at setting 7 for 10 minutes. Aliquot into a 3H plate.

Both test plates are placed into an Amersham 3H CAMP binding kit overnight, and harvested onto GF/B filters previously soaked in 0.5% PEI with a Skatron using 50 mM Tris HCl pH 7.4 at 4° C. Filtermats can be air-dried overnight then place in bags with 20 ml Betaplate scintillation cocktail and counted on a Betaplate counter for 60 sec per sample.

The in vitro activity of serotonin reuptake inhibitors, and the specificity for inhibition of serotonin as opposed to dopamine or norepinephrine reuptake, can be determined using rat synaptosomes or HEK-293 cells transfected with the human serotonin, dopamine or norepinephrine transporter, according to the following procedure adapted from those described by S. Snyder et al., (Molecular Pharmacology, 1971, 7, 66-80), D. T. Wong et al., (Biochemical Pharmacology, 1973, 22, 311-322), H. F. Bradford (Journal of Neurochemistry, 1969, 16, 675-684) and D. J. K. Balfour (EuroDean Journal of Pharmacology, 1973, 23, 19-26).

Synaptosomes: Male Sprague Dawley rats are decapitated and the brains rapidly removed. The cortex, hippocampi and corpus striata are dissected out and placed in ice cold sucrose buffer, 1 gram in 20 ml of buffer (the buffer is prepared using 320 mM sucrose containing 1 mg/ml glucose, 0.1 mM ethylenediamine tetraacetic acid (EDTA) adjusted to pH 7.4 with tris(hydroxymethyl)-aminomethane (TRIS) base). The tissues are homogenized in a glass homogenizing tube with a Teflon™ pestle at 350 rpm using a Potters homogenizer. The homogenate is centrifuged at 1000× g for 10 min. at 4° C. The resulting supernatant is recentrifuged at 17,000×g for 20 min. at 4° C. The final pellet is resuspended in an appropriate volume of sucrose buffer that yielded less than 10% uptake.

Cell Preparation: HEK-293 cells transfected with the human serotonin (5-HT), norepinephrine (NE) or dopamine (DA) transporter are grown in DMEM (Dulbecco's Modified Eagle Medium, Life Technologies Inc., 9800 Medical Center Dr., Gaithersburg, Md., catalog no. 11995-065)) supplemented with 10% dialyzed FBS (Fetal Bovine Serum, from Life Technologies, catalog no. 26300-053), 2 mM L-glutamine and 250 ug/ml G418 for the 5-HT and NE transporter or 2μg/ml puromycin for the DA transporter, for selection pressure. The cells are grown in Gibco triple flasks, harvested with Phosphate Buffered Saline (Life Technologies, catalog no. 14190-136) and diluted to an appropriate amount to yield less than 10% uptake.

Neurotransmitter Uptake Assay: The uptake assays are conducted in glass tubes containing 50 uL of solvent, inhibitor or 10 uM sertraline, desipramine or nomifensine for the 5-HT, NE or DA assay nonspecific uptake, respectively. Each tube contains 400 uL of [3H15-HT (5 nM final), [3H]NE (10 nM final) or [3H]DA (5 nM final) made up in modified Krebs solution containing 100 uM pargyline and glucose (1 mg/ml). The tubes are placed on ice and 50 uL of synaptosomes or cells is added to each tube. The tubes are then incubated at 370 C for 7 min. (5-HT, DA) or 10 min. (NE). The incubation is terminated by filtration (GF/B filters), using a 96-well Brandel Cell Harvester, the filters are washed with modified Krebs buffer and counted using either a Wallac Model 1214 or Wallac Beta Plate Model 1205 scintillation counter.

What is claimed is:

1. A method for treating a chemical dependency comprising administering an amount of a delta opioid receptor ligand and a serotonin reuptake inhibitor, said amounts being effective in said combination to treat said dependency, wherein said delta opioid receptor ligand is selected from the group consisting of:
a) a compound of the formula 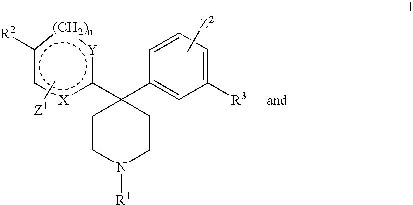
and
b) a compound of the formula 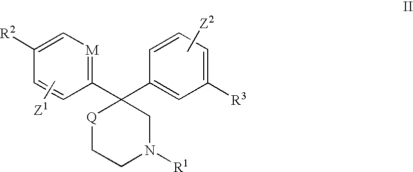
wherein
X and Y are selected, independently, from oxygen, nitrogen, sulfur and CH, with the proviso that the ring in compound I containing X and Y must be aromatic and with the proviso that X and Y cannot both be either oxygen or sulfur;
Q is oxygen or CH₂;
M is CH or N;
n is zero or one;
R¹ is hydrogen, (C₀-C₈)alkoxy-(C₀-C₈)alkyl-, wherein the total number of carbon atoms is eight or less, aryl, aryl-(C₁-C₈)alkyl-, heteroaryl, heteroaryl-(C₁-C₈)alkyl-, heterocyclic, heterocyclic-(C₁-C₈)alkyl, (C₃-C₇)cycloalkyl-, or (C₃-C₇)cycloalkyl-(C₁-C₈)alkyl, wherein said aryl and the aryl moiety of said aryl-(C₁-C₈)alkyl- are selected, independently, from phenyl and naphthyl, and wherein said heteroaryl and the heteroaryl moiety of said heteroaryl-(C₁-C₈)alkyl- are selected, independently, from pyrazinyl, benzofuranyl, quinolyl, isoquinolyl, benzothienyl, isobenzofuryl, pyrazolyl, indolyl, isoindolyl, benzimidazolyl, purinyl, carbazolyl, 1,2,5-thiadiazolyl, quinazolinyl, pyridazinyl, pyrazinyl, cinnolinyl, phthalazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidinyl, pyrazolopyrimidinyl, oxazolyl, oxadiazolyl, isoxazolyl, thiazolyl, isothiazolyl, furanyl, pyrazolyl, pyrrolyl, tetrazolyl, triazolyl, thienyl, imidazolyl, pyridinyl, and pyrimidinyl; and wherein said heterocyclic and the heterocyclic moiety of said heterocyclic-(C₁-C₈)alkyl- are selected from saturated or unsaturated nonaromatic monocyclic or bicyclic ring systems, wherein said monocyclic ring systems contain from four to seven ring carbon atoms, from one to three of which may optionally be replaced with O, N or S, and wherein said bicyclic ring systems contain from seven to twelve ring carbon atoms, from one to four of which may optionally be replaced with O, N or S; and wherein any of the aryl, heteroaryl or heterocyclic moieties of R¹ may optionally be substituted with from one to three substituents independently selected from halo, (C₁-C₆)alkyl optionally substituted with from one to seven fluorine atoms, phenyl, benzyl, hydroxy, acetyl, amino, cyano, nitro, (C₁-C₆)alkoxy, (C₁-C₆)alkylamino and [(C—CO₆)alkyl]₂amino, and wherein any of alkyl moieties in R¹ may optionally be substituted with from one to seven fluorine atoms;
R² is hydrogen, aryl, halo, heteroaryl, heterocyclic, SO₂R⁴, COR⁴, CONR⁵R⁶, COOR⁴, or C(OH)R⁵R⁶ wherein each of R⁴, R⁵ and R⁸ is defined, independently, as R¹ is defined above, or R⁵ and R⁶, together with the carbon or nitrogen to which they are both attached, form a three to seven membered saturated ring containing from zero to three heterocarbons selected, independently, from O, N and S, and wherein said aryl, heteroaryl, and heterocyclic are defined as such terms are defined above in the definition of R¹, and wherein any of the aryl, heteroaryl and heterocyclic moieties of R² may optionally be substituted with from one to three substituents, independently selected from halo, (C₁-C₆)alkyl optionally substituted with from one to seven fluorine atoms, phenyl, benzyl, hydroxy, acetyl, amino, cyano, nitro, (C₁-C₆)alkoxy optionally substituted with from one to seven fluorine atoms, (C₁-C₆)alkylamino and [(C₁-C₆)alkyl]₂amino;
R³ is hydroxy, -(C₁-C₆)alkyl-OH, —OC(=O)R⁷, —(C₁-C₆)alkyl-(C₁-C₆)alkoxy, NHSO₂R⁷, C(OH)R⁷R⁸, halo, or heteroaryl as defined for R¹ above or CONHR⁷, wherein R⁷ and R⁸ are the same or different and are selected from hydrogen, (C₁-C₄)alkyl, (C₁-C₄)alkoxy and (C₁-C₄)alkoxy-(C₁-C₄)alkyl having a total of 4 or less carbon atoms, and wherein any of the alkyl moieties of R⁷ and R⁸ may optionally be substituted with from one to seven fluorine atoms; and
Z¹ and Z² are independently hydrogen, halo or (C₁-C₅)alkyl;
with the proviso that there are no two adjacent ring oxygen atoms and no ring oxygen atom adjacent to either a ring nitrogen atom or a ring sulfur atom in any of the heterocyclic or heteroaryl moieties of formula I or II;
and the pharmaceutically acceptable salts of such compounds.

2. A method according to claim 1 wherein said delta opioid receptor ligand is a compound of the formula I wherein n is zero or one; X and Y are both nitrogen; R¹ is benzyl, cyclopropylmethyl, 2-pyridyl, 4-fluoro-2-pyridyl, pyrimidyl, 2-methylpentyl, 3-phenylpropyl, 2-ethoxyethyl or 3,5,5-trimethylhexyl; R² is CON(CH₂CH₃)₂, CON(CH₃)₂, CON(CH₂CH₃)CH₃, C(OH)(CH3)₂, C(OH)(CH₂CH₃)₂, 3,3-dimethyloxazoline, 3,3-diethyloxazoline, benzoxazole, tetrazole or 3,5-dimethylpyrazole; and R³ is OH, CONH₂, fluoro, bromo, chloro, iodo, or NHSO₂R⁷.

3. A method according to claim 1 wherein said delta opioid receptor ligand is a compound of the formula I wherein n is zero or one; X is nitrogen and Y is CH or oxygen; R¹ is benzyl, cyclopropylmethyl, 2-pyridyl, 4-fluoro-2-pyridyl, pyrimidyl, 2-methylpentyl, 3-phenylpropyl, 2-ethoxyethyl, 3,5,5-trimethylhexyl, allyl, cyclopropylmethyl, methyl, 2,2,2-trifluoroethyl, methallyl, isopropyl, 2-pyridinyl, 2-pyrimidinyl, or 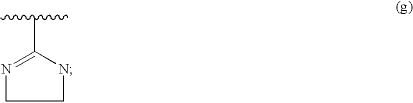
R² is CON(CH₂CH₃)₂, CON(CH₃)₂, CON(CH₂CH₃)CH₃, C(OH)(CH3)₂, C(OH)(CH₂CH₃)₂, 3,3-dimethyloxazoline, 3,3-diethyloxazoline, benzoxazole, tetrazole or 3,5-dimethylpyrazole; and R³ is OH, CONH₂, fluoro, bromo, chloro, iodo, or NHSO₂R⁷.

4. A method according to claim 3 wherein n is zero, Y is CH, and R³ is OH or CONH₂.

5. A method according to claim 1 wherein said opioid receptor ligand is of the formula II wherein R¹ is cyclopropylmethyl, 3-cyclohexylpropyl, 2-phenylethyl, 2-methylpentyl, p-methylbenzyl, 2,2,2-trifluoroethyl, or 1-methylpentyl, R² is diethyl amide, methyl ethyl amide, a diethyl carbinol, tetrazole, or pyrazole, and R³ is hydroxy, fluoro, CONH₂, NHSO₂CH₃, or methoxy.

6. A method according to claim 1 wherein said opioid receptor ligand is of the formula II and Q is CH₂, X is CH, R³ is OH, CONH₂, or fluoro, R² is selected from C(OH)(C₂H₆)₂, CON(C₂H₆)₂, CONCH₃(C₂H6) and the following cyclic groups: 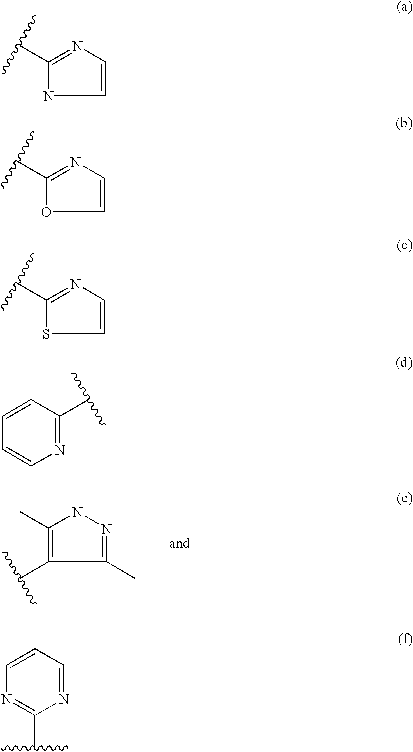
and wherein
Z¹ and Z² are selected, independently, from hydrogen and fluorine.

7. A method according to claim 1 wherein said opioid receptor ligand is selected from the group consisting of:
compounds of the formula II wherein Q is CH₂, M is N, R³ is OH, CONH₂, or fluoro, and R² is selected from C(OH)(C₂H₆)₂, CON(C₂H₆)₂ and one of cyclic groups (a)-(f): 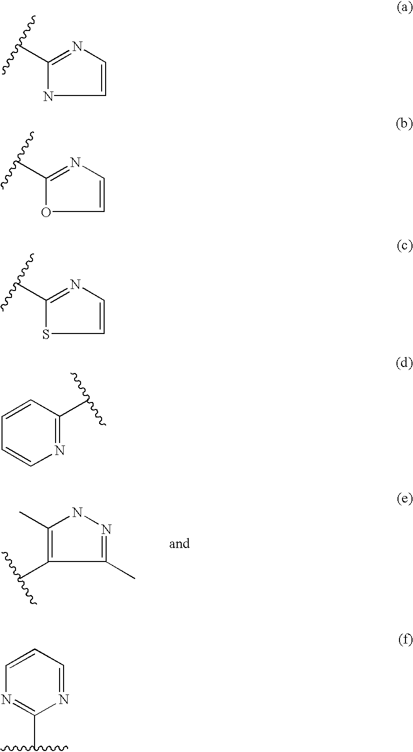
compounds of the formula II wherein Q is oxygen, M is N, R³ is OH, CONH₂, or fluoro, and R² is selected from C(OH)(C₂H₆)₂, CON(C₂H₆)₂ and one of cyclic groups (a)-(f) depicted above;
compounds of the formula II wherein Q is oxygen, M is CH, R³ is OH, CONH₂ or fluoro, Z¹ and Z² or selected, independently, from hydrogen and fluoro, and R¹ is selected from allyl, cyclopropylmethyl, methyl, methallyl, isopropyl, 2-pyridinyl, 2-pyrimidinyl and cyclic group (g) depicted above; and

8. A method according to claim 1 wherein said opioid receptor ligand is a compound of the formula II wherein Q is oxygen, M is N, R³ is OH, CONH₂ or fluoro, Z¹ and Z² or selected, independently, from hydrogen and fluoro, and R¹ is selected from allyl, cyclopropylmethyl, methyl, methallyl, isopropyl, 2-pyridinyl, 2-pyrimidinyl and cyclic group (g) depicted above.

9. A method of claim 1 wherein said serotonin reuptake ligand is selected from the group consisting of fluvoxamine sertraline, citalopram, fluoxetine, paroxetine, imipramine, zimelidine, vanlafaxine, and nefazodone.

10. A pharmaceutical composition for the treatment of a chemical dependency wherein composition comprises amounts of a delta opioid receptor ligand and a serotonin reuptake inhibitor, said amounts being effective in said combination to treat said dependency, wherein said delta opioid receptor ligand is selected from the group consisting of
a) a compound of the formula 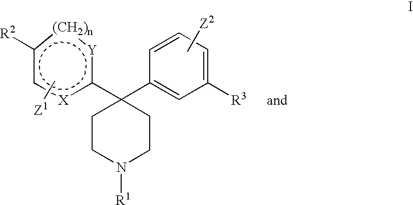
and
b) a compound of the formula 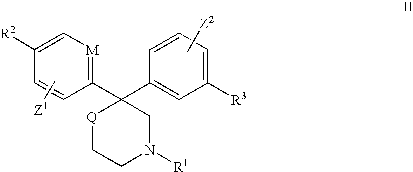
wherein
X and Y are selected, independently, from oxygen, nitrogen, sulfur and CH, with the proviso that the ring in compound I containing X and Y must be aromatic and with the proviso that X and Y cannot both be either oxygen or sulfur;
Q is oxygen or CH₂;
M is CH or N;
n is zero or one;
R¹ is hydrogen, (C₀-C₈)alkoxy-(C₁-C₈)alkyl-, wherein the total number of carbon atoms is eight or less, aryl, aryl-(C₁-C₈)alkyl-, heteroaryl, heteroaryl-(C₁-C₈)alkyl-, heterocyclic, heterocyclic-(C₁-C₈)alkyl, (C₃-C₇)cycloalkyl-, or (C₃-C₇)cycloalkyl-(C₁-C₈)alkyl, wherein said aryl and the aryl moiety of said aryl-(C₁-C₈)alkyl- are selected, independently, from phenyl and naphthyl, and wherein said heteroaryl and the heteroaryl moiety of said heteroaryl-(C₁-C₈)alkyl- are selected, independently, from pyrazinyl, benzofuranyl, quinolyl, isoquinolyl, benzothienyl, isobenzofuryl, pyrazolyl, indolyl, isoindolyl, benzimidazolyl, purinyl, carbazolyl, 1,2,5-thiadiazolyl, quinazolinyl, pyridazinyl, pyrazinyl, cinnolinyl, phthalazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidinyl, pyrazolopyrimidinyl, oxazolyl, oxadiazolyl, isoxazolyl, thiazolyl, isothiazolyl, furanyl, pyrazolyl, pyrrolyl, tetrazolyl, triazolyl, thienyl, imidazolyl, pyridinyl, and pyrimidinyl; and wherein said heterocyclic and the heterocyclic moiety of said heterocyclic-(C₁-C₈)alkyl- are selected from saturated or unsaturated nonaromatic monocyclic or bicyclic ring systems, wherein said monocyclic ring systems contain from four to seven ring carbon atoms, from one to three of which may optionally be replaced with O, N or S, and wherein said bicyclic ring systems contain from seven to twelve ring carbon atoms, from one to four of which may optionally be replaced with O, N or S; and wherein any of the aryl, heteroaryl or heterocyclic moieties of R¹ may optionally be substituted with from one to three substituents independently selected from halo, (C₁-C₆)alkyl optionally substituted with from one to seven fluorine atoms, phenyl, benzyl, hydroxy, acetyl, amino, cyano, nitro, (C₁-C₆)alkoxy, (C₁-C₆)alkylamino and [(C₁-C₆)alkyl]₂amino, and wherein any of alkyl moieties in R¹ may optionally be substituted with from one to seven fluorine atoms;
R²is hydrogen, aryl, halo, heteroaryl, heterocyclic, SO₂R⁴, COR⁴, CONR⁵R⁶, COOR⁴, or C(OH)R⁵R⁶ wherein each of R⁴, R⁵ and R⁶ is defined, independently, as R¹ is defined above, or R⁵ and R⁶, together with the carbon or nitrogen to which they are both attached, form a three to seven membered saturated ring containing from zero to three heterocarbons selected, independently, from O, N and S, and wherein said aryl, heteroaryl, and heterocyclic are defined as such terms are defined above in the definition of R¹, and wherein any of the aryl, heteroaryl and heterocyclic moieties of R² may optionally be substituted with from one to three substituents, independently selected from halo, (C₁-C₆)alkyl optionally substituted with from one to seven fluorine atoms, phenyl, benzyl, hydroxy, acetyl, amino, cyano, nitro, (C₁-C₆)alkoxy optionally substituted with from one to seven fluorine atoms, (C₁-C₆)alkylamino and ((C₁-C₆)alkyl]₂amino;
R³ is hydroxy, —(C₁-C₆)alkyl-OH, —OC(═O)R⁷, —(C₁-C₆)alkyl-(C₁-C₆)alkoxy, NHSO₂R⁷, C(OH)R⁷R⁸, halo, or heteroaryl as defined for R¹ above or CONHR⁷, wherein R⁷ and R⁸ are the same or different and are selected from hydrogen, (C₁-C₄)alkyl, (C₁-C₄)alkoxy and (C₁-C₄)alkoxy-(C₁-C₄)alkyl having a total of 4 or less carbon atoms, and wherein any of the alkyl moieties of R⁷ and R⁸ may optionally be substituted with from one to seven fluorine atoms; and
Z¹ and Z² are independently hydrogen, halo or (C₁-C₅)alkyl;
with the proviso that there are no two adjacent ring oxygen atoms and no ring oxygen atom adjacent to either a ring nitrogen atom or a ring sulfur atom in any of the heterocyclic or heteroaryl moieties of formula I;
and the pharmaceutically acceptable salts of such compounds.
